(19) United States
(12) Patent Application Publication
(10) Pub. No.: US 2023/0285717 A1
(43) Pub. Date: Sep. 14, 2023

(54) DUAL FUNCTION BALLOON CATHETER FOR CROSSING A LESION AND PERFORMING PERCUTANEOUS TRANSLUMINAL ANGIOPLASTY

(71) Applicant: BARD PERIPHERAL VASCULAR, INC., Tempe, AZ (US)

(72) Inventors: Amanda Young, Phoenix, AZ (US); Kyle Andrew Thomas, Phoenix, AZ (US); Keith Harris, Mesa, AZ (US); Clint Solomon, Gilbert, AZ (US); Genevieve Messina, Tempe, AZ (US)

(21) Appl. No.: 18/021,399

(22) PCT Filed: Aug. 31, 2020
(86) PCT No.: PCT/US2020/048699

§ 371 Date: Feb. 15, 2023

(51) Int. Cl. A61M 25/00; A61M 25/10

(52) U.S. Cl. CPC A61M 25/0069; A61M 25/005; A61M 25/1002; A61M 2025/1084; A61M 2025/1086; A61M 2025/1088; A61M 2025/109; A61M 2025/1093

(57) ABSTRACT

A catheter is provided for both crossing a lesion, such as a chronic total occlusion, in a vessel and performing percutaneous transluminal angioplasty thereupon. The catheter includes a shaft having a tapered tip adapted for being pushed into the lesion to advance through the lesion. An inflatable balloon is mounted on the shaft, which may be braided to provide enhanced pushability and kink-resistance. The inflatable balloon includes a proximal portion and a distal tapered portion connected to the tapered tip, and having a shallow taper. The taper of the distal tapered portion and the tapered tip may together create a continuous sloping transition from a distal end of the distal tapered portion to an adjacent portion of the tapered tip to facilitate crossing a lesion. The balloon may also be selectively inflated or deflated during crossing of the lesion, and ultimately used to compact the lesion when inflated.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Endovascular catheters are effective for treating many types of vascular disease, including by performing percutaneously transluminal angioplasty, or “PTA,” on a lesion obstructing blood flow in a vessel. Such a catheter may be introduced into a patient's vasculature and navigated via a guidewire to a treatment site. However, in some cases, the guidewire may encounter a stenosis completely blocking the vessel, which is known as a chronic total occlusion, or “CTO.”

In such a situation, the success of the procedure often depends on the ability to insert the guidewire through the chronic total occlusion. This may involve the use of a secondary device, such as a so-called “crossing” catheter, to establish guidewire access, such as by tunneling through the lesion using a cutter. Once such access is established, a different catheter is then used to perform a technique to treat the lesion, such as for example via PTA. The use of separate devices to cross the CTO and then treat the lesion complicates the procedure by requiring accessing the vasculature multiple times, and also increases the associated time and expense.

Accordingly, a need is identified for a single catheter than can perform the dual functions of both crossing a lesion, such as a CTO, and then performing percutaneous transluminal angioplasty or atherectomy on it. The device will be robust enough so that once it is used for such procedures, it may continue to be used on any other lesions that may need treatment.

### SUMMARY

According to one aspect of the disclosure, there is provided an apparatus for crossing a lesion in a vessel and performing percutaneous transluminal angioplasty on the lesion. The apparatus comprises a shaft including a guidewire lumen, an inflation lumen, and a tapered tip adapted for being pushed into the lesion. An inflatable balloon is mounted on the shaft and in communication with the inflation lumen, the inflatable balloon having a proximal portion, a barrel portion having a working surface for compacting the lesion, and a distal tapered portion. Together with the tapered tip, the distal tapered portion of the balloon provides a distal end portion of the shaft with a substantially continuous transition.

In one embodiment, at least a portion of the shaft proximal of the inflatable balloon is braided. A taper of the distal tapered portion of the inflatable balloon and the tapered tip create a continuous sloping transition from a proximal end of the distal tapered portion to a distal end of the tapered tip. This may be achieved by providing both the distal tapered portion and the tapered tip with a taper angle of less than about 20 degrees, such as for example 8-12 degrees, or about 10 degrees. The tapered tip may be elongated and extend to an end having a circular cross-section adapted for receiving and surrounding a guidewire. One or more scoring wires may optionally extend alongside the inflatable balloon. The shaft may include one or more radiopaque markers for identifying a working surface of the inflatable balloon when inflated. The inflatable balloon may be non-compliant, such as by including a layer of inelastic fibers, but may also be formed of elastic fibers, or as a composite balloon without fibers to provide a desired degree of compliance.

A further aspect of the disclosure relates to an apparatus for crossing a lesion in a vessel and performing percutaneous transluminal angioplasty on the lesion. The apparatus includes a shaft with a guidewire lumen, an inflation lumen, and a tapered tip having an end for being advanced into the lesion. An inflatable balloon is mounted on the shaft. More specifically, the inflatable balloon is attached to and forms a continuous sloping transition to the end of the tapered tip.

In one embodiment, the shaft is braided proximally of the inflatable balloon. The inflatable balloon may include a proximal tapered portion and a distal tapered portion. A taper of the distal tapered portion and the tapered tip together form the continuous sloping transition. The distal tapered portion and the tapered tip both have a taper angle of less than about 20 degrees. The inflatable balloon may be non-compliant.

In accordance with a further aspect of the disclosure, a balloon catheter for crossing a lesion in a vessel and performing percutaneous transluminal angioplasty on the lesion is provided. The balloon catheter comprises a shaft including at least a braided portion. The shaft includes a guidewire lumen, an inflation lumen, and a tapered tip adapted for being pushed into the lesion, the tip having a taper from a proximal end portion to a distal end thereof. An inflatable balloon is supported by the shaft and includes a distal tapered portion connected adjacent to the proximal end portion of the tip.

In one embodiment, the distal tapered portion has a first taper corresponding to a second taper of the tapered tip so as to provide the catheter with a substantially uniform taper along a distal end portion thereof. The tapered tip may be elongated, and may comprise a unitary part of the shaft. The inflatable balloon may be non-compliant, and the braided portion of the shaft may be proximal of the inflatable balloon.

This disclosure also relates to a method of crossing a lesion in a vessel and performing percutaneous transluminal angioplasty on the lesion. The method includes the steps of advancing a catheter including a tip having a continuous taper with a distal end portion of a balloon supported by a shaft of the catheter into the lesion, and compacting the lesion using the balloon. The advancing step may comprise advancing a guidewire passing through the shaft of the catheter into the lesion. The advancing step may further comprise successively inflating and deflating the inflated balloon.

### BRIEF DESCRIPTION OF THE DRAWINGS

The above and further advantages of the disclosure may be better understood by referring to the following description in conjunction with the accompanying drawings in which:

FIG. 1 is a partially cross-sectional side view of a balloon catheter according to the disclosure.

FIG. 1A is an enlarged partially cross-sectional side view of a distal end portion of the balloon catheter according to the disclosure.

FIG. 2 is a partial side-view of a non-compliant balloon for use in connection with the catheter of FIG. 1, according to the disclosure.

FIGS. 3, 3A, 3B, and 3C illustrate various shaft designs for use in the catheter of FIG. 1 according to the disclosure.

FIGS. 4, 5, 6, and 7 illustrate the possible use of the disclosed balloon catheter for crossing a lesion (such as a CTO) in a vessel and performing percutaneous transluminal angioplasty on the lesion.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the items depicted in the drawings may be combined into a single function.

### DETAILED DESCRIPTION

The present disclosure provides a single catheter than may be used to both cross a lesion, such as a CTO, and also perform percutaneous transluminal angioplasty. The device will be sufficiently robust so that once it is used for such procedures, it may continue to be used on any other lesions that may need treatment in a single intervention.

In the following detailed description, numerous specific details are set forth to provide a thorough understanding of the present disclosure. The disclosed embodiments may be practiced without these specific details. In other instances, well-known methods, procedures, components, or structures may not have been described in detail so as not to obscure the aspects of the present disclosure.

The present disclosure is directed to systems and methods for treatment of a vessel. The principles and operation of systems and methods of the disclosure may be better understood with reference to the drawings and accompanying descriptions.

The invention is not limited in its application to the details of construction and the arrangement of the components set forth in the following description or illustrated in the drawings.

The invention is capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination.

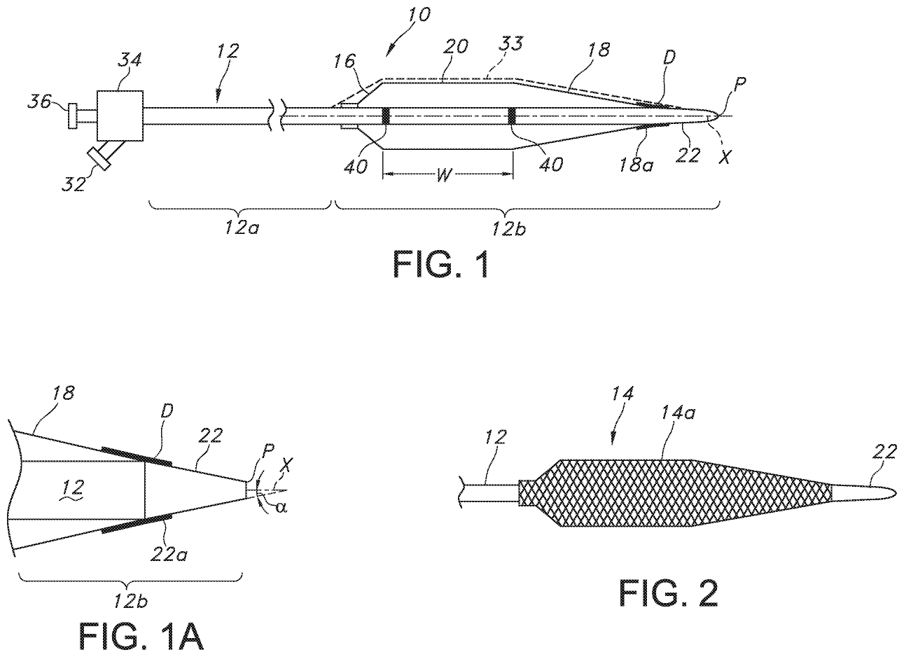

Referring now to FIGS. 1-3, a catheter 10 is provided for endovascular use. The catheter includes a shaft 12 having a proximal end portion 12a and a distal end portion 12b on which an inflatable balloon 14, is positioned. The balloon 14 includes a proximal tapered portion 16 sealed at one end to the shaft 12, a distal tapered portion 18 also sealed to the shaft 12, and a barrel portion 20 therebetween. The barrel portion 20 is generally cylindrical and, when the balloon 14 is inflated, this portion defines the working surface W for contacting and compressing a lesion during a PTA procedure.

The balloon 14 may be formed so as to be non-compliant, meaning it does not substantially increase in diameter once fully inflated as a result of increased fluid pressure being applied thereto. To achieve this condition, the balloon 14 may include inelastic fibers 14a, as shown in FIG. 2. The fibers 14a may be applied (such as to a base balloon or mandrel) in various forms, such as in an overlapping helical pattern, as shown in FIG. 2, in a braided or woven form, or as separate passes of longitudinal and transverse, or “hoop” fibers. Regardless of form, the applied fibers 14a may be covered by an outer layer or coating to provide the balloon 14 with a smooth and resilient outer surface. This type of non-compliant balloon 14 is fairly rigid and robust when inflated, which as understood below may aid in using it in connection with crossing a CTO. However, the balloon 14 may also be made semi-compliant, such as for example by using fibers 14a having a degree of elasticity or stretch. Alternatively, the balloon 14 may include no fibers, and comprise multiple, adhesively attached layers of materials to provide desirable strength or elasticity combinations.

The distal end of the catheter 10 includes a tip 22, which may be positioned distal of the distal tapered portion 18 of the balloon 14. The tip 22 may be elongated (e.g., having a length of greater than about 1 centimeter and a considerably smaller transverse dimension). As perhaps best understood from FIG. 1A, the tip 22 may taper in a distal direction from a maximum diameter D adjacent to a distalmost end of the balloon 14 to a relatively narrow point Pat the distal end of the catheter 10. The taper may be continuous and/or uniform, and may extend at an angle a relative to a longitudinal axis X of less than about 20 degrees, such as for example between 1-20 degrees and, more specifically, from about 10 to about 20 degrees. The tip 22 may be formed of an atraumatic material (i.e., a soft and flexible material, which may comprise a material such as, for example, a plastic resin, polyethylene, polyvinylchloride, silicone, or Teflon) to prevent damage to the vessel, and includes an opening at the point P having a circular cross-section through which the guidewire 30 may pass and be held in position during the catheter advance through a lesion. The tip 22 may be integrally connected to or unitarily formed with the distal end portion 12b of the shaft 12.

From FIGS. 1, 1A and 2, it can be appreciated that the distal tapered portion 18 of the balloon 14 in an inflated state has a fairly gradual or shallow taper in the longitudinal direction, with the width lessening from a proximal end to a distal end of the distal tapered portion 18. The arrangement may be such that the taper angle of the distal tapered portion 18 of the balloon 14 and the tapered tip 22 correspond or are substantially the same (that is, the same angle a is followed uniformly in the longitudinal direction), but the taper angles of the distal tapered portion and tapered tip 22 could be different. In any case, a continuous sloping transition is created from a proximal end of the distal tapered portion 18 to an adjacent distal end portion 22a of the tapered tip 22 to which the balloon 14 is attached, and ultimately to the point P. By “continuous sloping transition,” it is meant that the angle of the taper created along the distal end portion 12b of the shaft 12 is substantially continuous from the proximal end of the distal tapered portion 18 (corresponding to the transition from the barrel portion 20) to adjacent the point P at the distal end of the tip 22, but does not mean the angle cannot vary by several degrees along the transition while still being continuously tapered.

As can be further appreciated, this continuously sloping transition feature provides the catheter 10 with a gradual, but substantially continuously increasing diameter from the point P at the distal end of the tip 22 to at least the distal end of the barrel portion 20, when viewed in the proximal direction. This tapering is generally smooth and substantially uninterrupted, except possibly where a neck 18a at the distal tapered portion 18 of the balloon 14 overlaps with and is attached to shaft 12 or tapered tip 22 (as shown in FIG. 1A). The attachment may be achieved using known bonding techniques, such as welding, adhesive, or the like (and the bond may possibly covered by fibers, if present).

The taper angle of the distal tapered portion 18 of the balloon 14 and the tip 22 relative to the longitudinal axis X may be shallow, such as less than about 20 degrees. For example, the taper angle may be about 1-20 degrees or less, such as for example anywhere between about 10 to about 20 degrees (the value of the taper angle in the illustrated embodiment is, for example, about 8-12 degrees, or about 10 degrees). In any case, the taper angle of the distal tapered portion 18 may be less than the taper angle of the proximal tapered portion 16 of the balloon 14, which is typically 20-30 degrees or more.

As can be appreciated, this means that the distal tapered portion 18 is elongated (that is, longer than it is wide), and thus extends along a greater portion of the shaft 12 than the proximal tapered portion 16 (such as 2-3 times more). While the angles of the tapered tip 22 and the distal tapered portion 18 of the balloon 14 substantially match, this provides the distal tapered portion 18 of the catheter 10 with a substantially uniform taper. As can be understood from FIGS. 1 and 2, this arrangement also provides the balloon 14 with asymmetry transverse to the longitudinal axis X.

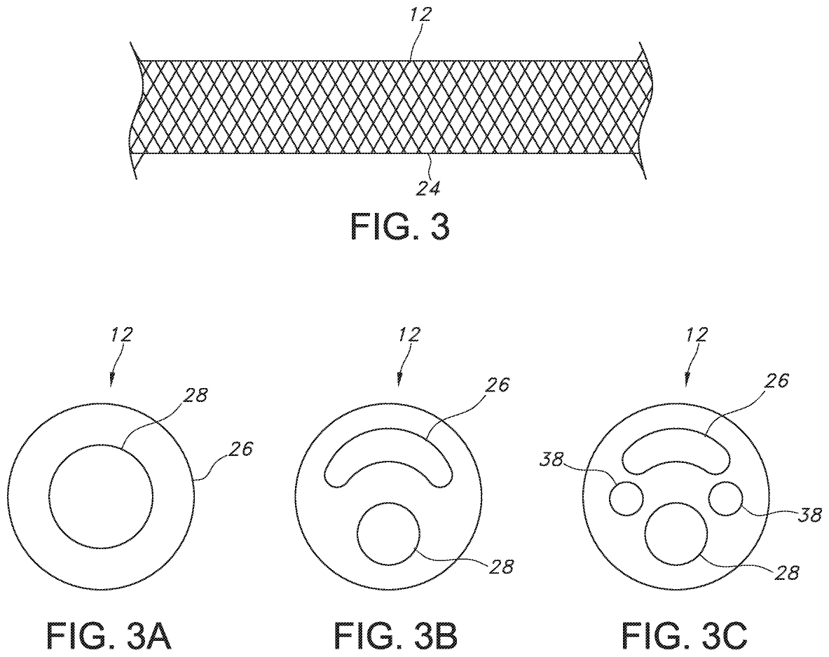

With reference to FIG. 3, the shaft 12 may be adapted to improve the pushability of the catheter 10. In one example, this may be achieved by applying a braiding 24 at least a portion of the shaft 12, such as along a proximal end portion 12a. The shaft 12 may comprise a polymer tube formed by extrusion (including possibly co-extrusion of different materials), to which the braiding 24 is applied. For example, the braiding 24 may be applied to the shaft 12 proximally of the balloon 14, or up to and including the tip 22. This braiding 24 may comprise a thin metal wire, which once applied enhances the tensile strength of the tube forming the shaft 12. This retains sufficient flexibility for navigating through tortuous anatomy, while allowing for a significant longitudinal force to be placed on the catheter 10, such as while being pushed for crossing a CTO, while reducing any tendency for buckling or kinking. The braiding 24 may be externally applied, as shown, or may be internal to the shaft 12, including by embedding it into the material forming the polymer tube used as the basis for the shaft 12. Pushability may also be enhanced in other ways, as is known in the art, such as by increasing the wall thickness of the tube forming shaft 12, with a consequence that inflation/deflation times may be increased as a result, which of course may impact performance, especially when multiple inflation events are necessitated.

The shaft 12 may have one of a variety of cross-sectional configurations. For example, as shown in FIG. 3A, the shaft 12 may be of a coaxial design, and include a first, outer inflation lumen 26 for transmitting a fluid for inflating or deflating the balloon 14, and a second, inner lumen 28 (which may be formed in a separate tube passing through balloon 14 and connected to tip 22) for passing a guidewire 30 through the catheter 10 to form an “over the wire” (OTW) arrangement. The inflation lumen 26 may communicate with an inflation port 32 located on a hub 34 connected to the shaft 12. The guidewire lumen 28 may communicate with a guidewire port 36 also associated with the hub 34.

Alternatively, as shown in FIG. 3B, the shaft 12 may comprise a “dual lumen” arrangement. In this approach, the inflation lumen 26 and guidewire lumen 28 are located side-by-side, rather than being on a common axis in the FIG. 3A version. In this embodiment, communication is still established with the respective ports 32, 36 in the hub 34.

Optionally, the catheter 10 may include one or more external scoring wires 33 extending alongside the balloon 14 for use in scoring or cutting the lesion on expansion. In such case, the shaft 12 may include additional lumen(s) 38 in the shaft 12 for receiving the proximal portions of these scoring wires, as shown in FIG. 3C. The wire or wires 33 may be fixed against movement longitudinally, or may optionally be designed to move longitudinally as the balloon 14 expands, such as by attaching the wires to a proximal end portion 12a of the shaft 12 via a spring or like biasing mechanism.

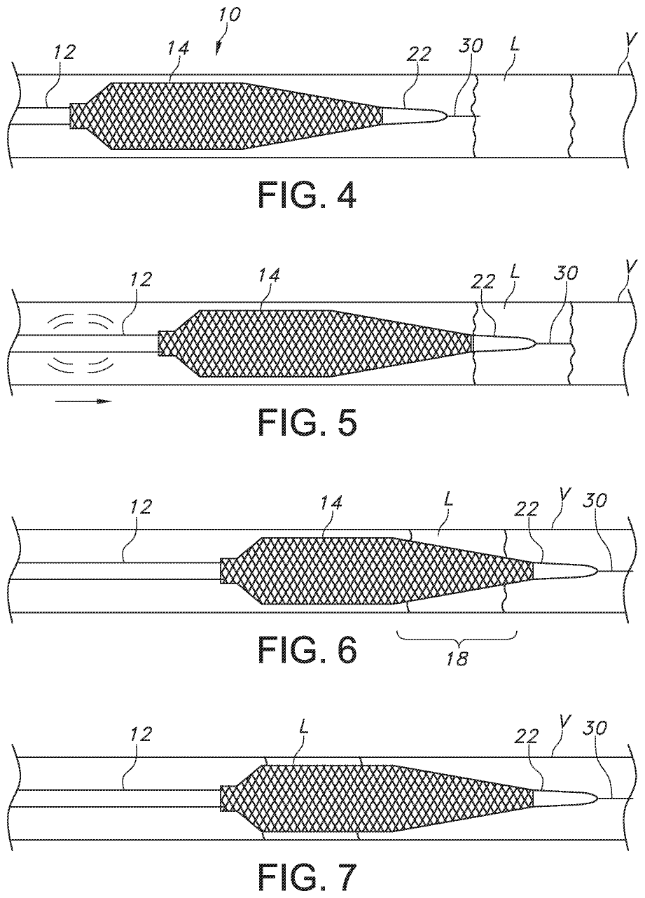

Turning to FIGS. 4, 5, 6, and 7 the use of the catheter 10 in both crossing a lesion, such as a CTO, and then performing a PTA procedure, is described. Once a guidewire 30 is advanced to the site of a lesion L in a vessel V, with the balloon 14 in a deflated, folded condition, the catheter 10 may be guided along the guidewire to a position adjacent the lesion L and the balloon 14 inflated, as shown in FIG. 4. Positioning of the balloon 14 may be aided by the use of fluoroscopy, and one or more suitable radiopaque markers 40, such as for example metal bands, may be provided on the shaft 12 for this purpose (see FIG. 1). The marker(s) 40 may be positioned so as to mark the edges or boundaries of the working surface W of the balloon 14.

In a typical CTO situation, the guidewire 30 alone may be unable to fully penetrate the lesion L to gain access to a distal end thereof. In such case, the tapered tip 22 of the catheter 10 may be advanced along the guidewire 30 and into the lesion L to help create a path for advance, as shown in FIG. 5. As can be appreciated, the enhanced pushability provided by the at least partially braided shaft 12 aids pushing the tip 22 through the lesion without buckling or kinking. Likewise, the inflated condition of the balloon 14 serves to help keep the guidewire 30 centered in the vessel V, and helps to avoid it from going subintimal.

The advance may continue, as shown in FIG. 6, with the tapered distal end portion 18 of the inflated balloon 14 serving to gradually expand the lesion L as the advance is made. Depending on the circumstances, this may require successively altering the diameter of the balloon 14, such as by at least partially deflating it to progressively move through the lesion L via the tapered tip 22, and then re-inflating it to create a degree of transverse compaction in the lesion (such as via the tapered distal portion 18) and further establish a path of travel therethrough. This process may be repeated as necessary to cross the lesion and place the barrel portion 20 of the balloon 14 in a position for fully compacting the lesion to the maximum extent possible. In the case of a non-compliant, fiber-based version, as shown, the balloon 14 is sufficiently robust to experience multiple inflations and deflations and advance through the lesion L, without degradation or unintended deflation (rupture).

Ultimately, the barrel portion 20 of the balloon 14 may become positioned fully within the lesion L. When expanded to the maximum diameter, if not already done, the balloon 14 compacts the lesion L, thus recanalizing and clearing the vessel V of the obstruction (and may also serve to slice or score the lesion when one or more cutters, such as scoring wire(s) 33, are optionally provided). The balloon 14 may also optionally carry a treatment agent, such as a drug, for application to the compacted lesion L.

Once the PTA procedure is completed, the balloon 14 may then be deflated, and optionally refolded. With aid of guidewire 30, the catheter 10 may then be guided to another location in the vessel V or vasculature for crossing a lesion or performing PTA, with the balloon 14 design again being sufficiently robust to again withstand reinflation and lesion compaction. Additionally, or alternatively, the catheter 10 may simply be withdrawn from the vessel V once the crossing and compaction of a single lesion is completed.

The catheter 10 may of course be provided in a variety of diameters depending on the use to which it may be put in the vasculature. To facilitate use in the vasculature below-the-knee (BTK), the catheter 10 may be made so as to have a small size. For example, the catheter 10 may be 5 French (approximately 5.24 mm in circumference, or 1.667 mm in outer diameter). Other sizes may be used depending on the application.

Summarizing, this disclosure may be considered to relate to the following items:
1. An apparatus for crossing a lesion in a vessel and performing percutaneous transluminal angioplasty on the lesion, comprising:
   a shaft including a guidewire lumen, an inflation lumen, and a tapered tip adapted for being pushed into the lesion; and
   an inflatable balloon mounted on the shaft and in communication with the inflation lumen, the inflatable balloon having a proximal portion, a barrel portion having a working surface for compacting the lesion, and a distal tapered portion together with the tapered tip providing a distal end portion of the shaft with a substantially continuous transition.
2. The apparatus of item 1, wherein at least a portion of the shaft proximal of the inflatable balloon is braided.
3. The apparatus of item 1 or item 2, wherein a taper of the distal tapered portion of the inflatable balloon and the tapered tip create a continuous sloping transition from a proximal end of the distal tapered portion to a distal end of the tapered tip.
4. The apparatus of any of items 1-3, wherein the distal tapered portion and the tapered tip both have a taper angle of less than about 20 degrees.
5. The apparatus of item 4, wherein the taper angle is about 10 degrees.
6. The apparatus of any of items 1-5, wherein the tapered tip is elongated and extends to an end having a circular cross-section adapted for receiving and surrounding a guidewire.
7. The apparatus of any of items 1-6, further including one or more scoring wires extending alongside the inflatable balloon.
8. The apparatus of any of items 1-7, wherein the shaft includes one or more radiopaque markers for identifying a working surface of the inflatable balloon when inflated.
9. The apparatus of any of items 1-8, wherein the inflatable balloon is non-compliant.
10. An apparatus for crossing a lesion in a vessel and performing percutaneous transluminal angioplasty on the lesion, comprising:
   a shaft including a guidewire lumen, an inflation lumen, and a tapered tip having an end for being advanced into the lesion; and
   an inflatable balloon mounted on the shaft, the inflatable balloon attached to and forming a continuous sloping transition to the end of the tapered tip.
11. The apparatus of item 10, wherein the shaft is braided proximally of the inflatable balloon.
12. The apparatus of item 10 or item 11, wherein the inflatable balloon includes a proximal tapered portion and a distal tapered portion, a taper of the distal tapered portion and the tapered tip together forming the continuous sloping transition.
13. The apparatus of any of items 10-12, wherein the distal tapered portion and the tapered tip both have a taper angle of less than about 20 degrees.
14. The apparatus of any of items 10-13, wherein the inflatable balloon is non-compliant.
15. A balloon catheter for crossing a lesion in a vessel and performing percutaneous transluminal angioplasty on the lesion, comprising:
   a shaft including at least a braided portion, the shaft including a guidewire lumen, an inflation lumen, and a tapered tip adapted for being pushed into the lesion, the tip having a taper from a proximal end portion to a distal end thereof; and
   an inflatable balloon supported by the shaft and having a distal tapered portion connected adjacent to the proximal end portion of the tip.
16. The balloon catheter of item 15, wherein the distal tapered portion has a first taper corresponding to a second taper of the tapered tip so as to provide the catheter with a substantially uniform taper along a distal end portion thereof.
17. The balloon catheter of item 15 or item 16, wherein the tapered tip is elongated.
18. The balloon catheter of any of items 15-17, wherein the tapered tip is a unitary part of the shaft.
19. The balloon catheter of any of items 15-18, wherein the inflatable balloon is non-compliant.
20. The balloon catheter of any of items 15-19, wherein the braided portion of the shaft is proximal of the inflatable balloon.
21. A method of crossing a lesion in a vessel and performing percutaneous transluminal angioplasty on the lesion, comprising:
   advancing a catheter including a tip having a continuous taper with a distal end portion of a balloon supported by a shaft of the catheter into the lesion; and
   compacting the lesion using the balloon.
22. The method of item 21, wherein the advancing step includes advancing a guidewire passing through the shaft of the catheter into the lesion.
23. The method of item 21 or item 22, wherein the advancing step further comprises successively inflating and deflating the inflated balloon.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” or “approximately,” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains”, and are inclusive or open-ended terms that specifies the presence of what follows e.g. component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the appended claims' spirit and scope. All publications, patents and patent applications mentioned in this specification are herein incorporated in their entirety by reference into the specification, to the same extent as if each individual publication, patent or patent application was specifically and individually indicated to be incorporated herein by reference. In addition, or identification of any reference in this application shall not be construed as an admission that such reference is available as prior art to the present disclosure.

1. An apparatus for crossing a lesion in a vessel and performing percutaneous transluminal angioplasty on the lesion, comprising:
a shaft including a guidewire lumen, an inflation lumen, and a tapered tip for being pushed into the lesion; and
an inflatable balloon mounted on the shaft and in communication with the inflation lumen, the inflatable balloon having a proximal portion, a barrel portion having a working surface for compacting the lesion, and a distal tapered portion together with the tapered tip providing a distal end portion of the shaft with a substantially continuous transition.

2. The apparatus of claim 1, wherein at least a portion of the shaft proximal of the inflatable balloon is braided.

3. The apparatus of claim 1, wherein a taper of the distal tapered portion of the inflatable balloon and the tapered tip create a continuous sloping transition from a proximal end of the distal tapered portion to a distal end of the tapered tip.

4. The apparatus of claim 1, wherein the distal tapered portion and the tapered tip each have a taper angle of less than about 20 degrees.

5. The apparatus of claim 4, wherein the taper angle is 8-12 degrees.

6. The apparatus of claim 1, wherein the tapered tip is elongated and extends to an end having a circular cross-section adapted for receiving and surrounding a guidewire.

7. The apparatus of claim 1, further including one or more scoring wires extending alongside the inflatable balloon.

8. The apparatus of claim 1, wherein the shaft includes one or more radiopaque markers for identifying the working surface of the inflatable balloon when inflated.

9. The apparatus of claim 1, wherein the inflatable balloon is non-compliant.

10. An apparatus for crossing a lesion in a vessel and performing percutaneous transluminal angioplasty on the lesion, comprising:
a shaft including a guidewire lumen, an inflation lumen, and a tapered tip having an end for being advanced into the lesion; and
an inflatable balloon mounted on the shaft, the inflatable balloon attached to and forming a continuous sloping transition to the end of the tapered tip.

11. The apparatus of claim 10, wherein the shaft is braided proximally of the inflatable balloon.

12. The apparatus of claim 10, wherein the inflatable balloon includes a proximal tapered portion and a distal tapered portion, a taper of the distal tapered portion and the tapered tip together forming the continuous sloping transition.

13. The apparatus of claim 12, wherein the distal tapered portion and the tapered tip both have a taper angle of less than 20 degrees.

14. The apparatus of claim 10, wherein the inflatable balloon is non-compliant.

15. A balloon catheter for crossing a lesion in a vessel and performing percutaneous transluminal angioplasty on the lesion, comprising:
a shaft including at least a braided portion, the shaft including a guidewire lumen, an inflation lumen, and a tapered tip for being pushed into the lesion, the tip having a taper from a proximal end portion to a distal end thereof; and
an inflatable balloon supported by the shaft and having a distal tapered portion connected adjacent to the proximal end portion of the tip.

16. The balloon catheter of claim 15, wherein the distal tapered portion has a first taper corresponding to a second taper of the tapered tip so as to provide the catheter with a substantially uniform taper along a distal end portion thereof.

17. The balloon catheter of claim 15, wherein the tapered tip is elongated.

18. The balloon catheter of claim 15, wherein the tapered tip is a unitary part of the shaft.

19. The balloon catheter of claim 15, wherein the inflatable balloon is non-compliant.

20. The balloon catheter of claim 15, wherein the braided portion of the shaft is proximal of the inflatable balloon.

21-23. (canceled)
